(19) United States
(12) Patent Application Publication
(10) Pub. No.: US 2022/0265569 A1
(43) Pub. Date: Aug. 25, 2022

(54) PERMEANT DELIVERY PATCH VIA A FORMED PATHWAY

(71) Applicant: PASSPORT TECHNOLOGIES, INC., San Diego, CA (US)

(72) Inventors: Shohei Horie, San Diego, CA (US); Masato Nishimura, Oaska (JP); Joe Hua, Rosemead, CA (US); Sunny Kumar, Chatham, NJ (US); Hirotoshi Adachi, San Diego, CA (US)

(21) Appl. No.: 17/623,020

(22) PCT Filed: Jun. 24, 2020
(86) PCT No.: PCT/US2020/039426

§ 371 Date: Dec. 27, 2021

Related U.S. Application Data
(60) Provisional application No. 62/868,651, filed on Jun. 28, 2019.

(51) Int. Cl. A61K 9/70; A61K 47/26; A61K 47/12; A61K 47/40; A61K 47/14; A61K 47/18

(52) U.S. Cl. CPC A61K 9/7092; A61K 47/26; A61K 47/12; A61K 47/40; A61K 47/14; A61K 47/18

(57) ABSTRACT

Thin solid tablet compositions containing an active permeant can be used in methods for administering the permeant to a subject. The thin solid tablet can be incorporated into a patch. The patch can be used to administer the permeant, such as a drug and an excipient, to the subject by transdermal microporation.

### BACKGROUND

### Field

This application relates to compositions and methods for transdermal drug delivery, and in particular to thin solid tablet compositions containing an active permeant and methods for administering the permeant to a subject by transdermal microporation.

### Description

Passive transdermal drug delivery is a convenient and effective way to administer a variety of therapeutics. This route of administration is both noninvasive and produces steady drug delivery over an extended period of time. While conventional transdermal systems (such as drug patches) have demonstrated the benefits of delivering drugs via the skin, they only work for an extremely limited number of drugs. This is because millions of dead skin cells form a protective barrier on the surface of the skin (the stratum corneum) that prevents most therapeutic molecules from passing into the skin.

The stratum corneum is chiefly responsible for the barrier properties of skin. Thus, it is this layer that presents the greatest barrier to transdermal flux of drugs or other molecules into the body and of analytes out of the body. The stratum corneum, the outer homy layer of the skin, is a complex structure of compact keratinized cell remnants separated by lipid domains. Compared to the oral or gastric mucosa, the stratum corneum is much less permeable to molecules either external or internal to the body. The stratum corneum is formed from keratinocytes, which comprise the majority of epidermal cells that lose their nuclei and become corneocytes. These dead cells comprise the stratum corneum, which has a thickness of only about 10-30 microns and protects the body from invasion by exogenous substances and the outward migration of endogenous fluids and dissolved molecules. The stratum corneum is continuously renewed by shedding of corneum cells during desquamination and the formation of new corneum cells by the keratinization process.

Historically, the majority of drugs have been delivered orally or by injection. However, neither the oral or injection route is well-suited for continual delivery of drugs over an extended period of time. Further, the injection method of administration is inconvenient and uncomfortable; additionally, needles continue to pose a hazard after their use. Therefore, transdermal drug delivery to the body has been a popular and efficacious method for delivering a limited number of permeants into an organism.

Passive transdermal patches are typically limited to lipid-soluble drugs with a molecular weight of less than 500 daltons. To enhance transdermal drug delivery, there are known methods for increasing the permeability of the skin to drugs. For example, U.S. Pat. No. 8,116,860 describes transdermal permeant delivery systems and methods that painlessly create aqueous micropores in the stratum corneum within a few milliseconds. These aqueous channels enable water-soluble drugs to flow from a transdermal patch, enter the viable epidermis and then the systemic circulation. The patch may be formulated to provide for bolus or sustained transdermal delivery.

Transdermal permeant delivery systems are being developed under the PASSPORT tradename, The PASSPORT system comprises a reusable handheld applicator and a single-use porator with drug patch. Pressing the activation button of the applicator releases a pulse of energy to the porator. The rapid conduction of this energy into the surface of the skin painlessly ablates the stratum corneum under each filament to create the microchannels. A simple transdermal patch is then applied to the ablated skin and drug delivery begins.

However, despite the widespread availability of such systems and the significant benefits they provide, there remains a need for improved compositions and methods for transdermal drug delivery.

### SUMMARY

An embodiment provides a composition for delivery of a permeant through a pathway in a biological membrane of a subject comprising:

at least one thin solid tablet having an area density of more than 30 mg/cm² and less than 400 mg/cm²;

wherein the thin solid tablet comprises at least one permeant; and

wherein at least a portion of the permeant is soluble in biological moisture received from at least one pathway formed through the biological membrane of the subject.

Another embodiment provides a patch for delivering an agent via at least one formed pathway through a biological membrane of a subject, the patch comprising a composition that comprises a thin solid tablet as described elsewhere herein.

Another embodiment provides a method of treating a patient comprising:

opening at least one channel in the patient's skin;

applying a patch as described elsewhere herein to the patient's skin to thereby contact the at least one thin tablet with the channel; and

maintaining the at least one thin tablet in contact with the patient's skin for a period of time effective to:

(a) at least partially dissolve the permeant in biological moisture received from the pathway; and

(b) deliver a therapeutically effective amount of the resulting dissolved. permeant through the pathway to the patient.

Another embodiment provides a method of delivering a permeant through a pathway in a biological membrane of a subject comprising applying a patch as described. elsewhere herein to the patient's skin.

These and other embodiments are described in greater detail below.

### BRIEF DESCRIPTION OF THE DRAWINGS

FIG. 1A schematically illustrates a patch configuration having a thin solid tablet in a tablet layer, a backing layer over the tablet layer, and a release liner layer under the tablet layer. An option is illustrated in which the thin solid tablet is positioned in a cavity formed in the backing layer. The backing may include an adhesive (not shown) to maintain the position of the thin solid layer in the cavity.

FIG. 1B schematically illustrates a patch configuration having a thin solid tablet in a tablet layer, a backing layer over the tablet layer, a release liner layer under the tablet layer, and a cover under the tablet layer and over the release liner layer. Optionally, the cover can be a drug release control membrane. As in FIG. 1A, an option is illustrated in which the thin solid tablet is positioned in a cavity formed in the hacking layer. The backing may include an adhesive not shown) to maintain the position of the thin solid layer in the cavity.

FIG. 2 schematically illustrates a patch configuration having a thin solid tablet in a tablet layer, a backing layer over the tablet layer, an optional cover under the tablet layer, and a spacer layer between the backing layer and the cover layer. Optionally (not shown), the patch can further comprise a release liner layer positioned under the cover (or under the tablet layer when the optional cover is absent) in the manner indicated in FIGS. 1A and 1B. The spacer layer is laterally adjacent to the tablet and configured to maintain a separation distance between the backing layer and the cover and the optional release liner layer. Optionally, the cover can be a drug release control membrane.

FIG. 3 schematically illustrates a patch configuration similar to that of FIG. 2 except that the tablet layer contains two thin solid tablets (or, optionally, a thin solid. tablet and a film coated on the tablet) that are vertically adjacent to one another. The cover is optional. As in FIG. 2, the patch can optionally further comprise a release liner layer (not shown) positioned under the cover in the manner indicated in FIG. 4. Optionally, the cover can be a drug release control membrane.

FIG. 4 schematically illustrates a patch configuration similar to that of FIG. 3 except that the two thin solid tablets in the tablet layer are laterally adjacent to one another. Optionally, the cover can be a drug release control membrane.

FIG. 5 illustrates the pharmacokinetic (PK) profile of methylnaltrexone bromide released from a first thin solid tablet in a patch having a configuration as illustrated in FIG. 3.

FIG. 6 illustrates a comparative PK profile of methylnaltrexone bromide released from a comparative dry patch (dispensing type). The amount of methylnaltrexone bromide released was much less than the amounts released using the various configurations summarized in FIG. 5.

FIG. 7 illustrates the PK profile of aripiprazole released from a film coated first thin solid tablet in a patch having a. configuration as illustrated in FIG. 3 (with cover). The first thin solid tablet contained solubilizer (pH control agent and cyclodextrin) in addition to the aripiprazole.

FIG. 8 illustrates the PK profile of aripiprazole released from a film coated first thin solid tablet in a patch having a configuration as illustrated in FIG. 3 (with and without cover). The first thin solid tablet contained solubilizer (pH control agent and cyclodextrin) in addition to the aripiprazole.

FIG. 9 illustrates the PK profile of aripiprazole released from a film coated first thin solid tablet in a patch having a configuration as illustrated in FIG. 3 (with and without cover). The first thin solid tablet contained solubilizer (pH control agent and cyclodextrin) in addition to the aripiprazole.

FIG. 10 illustrates the PK profile of aripiprazole released from a first thin solid tablet (Groups 2 and 5) as compared to release from a first thin solid tablet in combination with a second thin solid tablet (Group 4), in patches having a configuration as illustrated in FIG. 3 (with cover). The first thin solid tablet(s) contained solubilizer (pH control agent and cyclodextrin) in addition to the aripiprazole. The pharmacokinetic profile illustrates sustained delivery.

FIG. 11 describes the (partial) patch configurations and ingredients for the patches of FIG. 10.

FIG. 12 illustrates the PK profile of sumatriptan released from a comparative dry patch. A color change was observed to occur during storage, indicating interaction between the sumatriptan and ascorbic acid.

FIG. 13 illustrates the PK profile of sumatriptan released from a film layer on a thin solid tablet in patches having a configuration as illustrated in FIG. 3. The thin solid tablet contained ascorbic acid; the film layer did not. The separation of the ascorbic acid from the sumatriptan enhanced the stability of the formulation.

### DETAILED DESCRIPTION

The present invention can be understood more readily by reference to the following detailed description, examples, drawing, and claims, and their previous and following descriptions. However, before the present devices, systems, and/or methods are disclosed and described, it is to be understood that this invention is not limited to the specific devices, systems, and/or methods disclosed unless otherwise specified. It is also to be understood that the terminology used herein is for the purpose of describing particular aspects only and is not necessarily intended to be limiting.

This description is provided as an enabling teaching of the invention. To this end, those skilled in the relevant art will recognize and appreciate that many changes can be made to the various aspects of the invention described herein, while still obtaining beneficial results. It will also be apparent that some of the desired benefits can be obtained by selecting some of the features described herein without utilizing other features. Accordingly, those who work in the art will recognize that many modifications and adaptations to the present description are possible and can even be desirable in certain circumstances and are a part of the present invention. Thus, this description is provided as illustrative of certain principles of the present invention and not in limitation thereof.

### Definitions

As used throughout, the singular forms “a,” “an,” and “the” include plural referents unless the context clearly dictates otherwise. Thus, for example, reference to “a filament” can include two or more such filaments unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein, a “tissue membrane” can be any one or more epidermal layers of a subject. For example, in one aspect, the tissue membrane is a skin layer that includes the outermost layer of the skin, i.e., the stratum corneum. In an alternative aspect, a skin layer can include one or more backing layers of the epidermis, commonly identified as stratum granulosum, stratum malpighii, and stratum germinativum layers. It will be appreciated by one of ordinary skill in the art that there is essentially little or no resistance to transport or to absorption of a permeant through the backing layers of the epidermis. Therefore, in one aspect, an at least one formed pathway in a skin layer of a subject is a pathway in the stratum corneum layer of a subject. Further, as used herein, “stratum corneum” refers to the outermost layer of the skin, typically containing from about 15 to about 20 layers of cells in various stages of drying out. The stratum corneum provides a barrier to the loss of water from inside the body to the external environment and from attack from the external environment to the interior of the body. Still further, as used herein, “tissue membrane” can refer to an aggregate of cells of a particular kind, together with their intercellular substance, that forms a structural material. In various embodiments at least one surface of the tissue membrane is accessible to one or more of the poration devices and/or permeant compositions described herein. As noted above, the preferred tissue membrane is the skin. Other tissues suitable for use with such devices and compositions include mucosal tissue and soft organs.

As used herein, the term, “subcutaneous fluid” can include, without limitation, moisture, plasma, blood, one or more proteins, interstitial fluid, and any combination thereof. In one aspect, a subcutaneous fluid according to this description is a moisture source comprising water.

As used herein, “poration,” “microporation,” or any such similar term means the formation of a small hole or crevice (subsequently also referred to as a “micropore”) in or through the tissue or biological membrane, such as skin or mucous membrane, or the outer layer of an organism to lessen the barrier properties of this biological membrane for the passage of at least one permeant from one side of the biological membrane to the other for select purposes. Preferably the hole or “micropore” so formed is approximately 1-1000 microns in diameter and extends into the biological membrane sufficiently to break the barrier properties of the stratum corneum without adversely affecting the underlying tissues, It is to be understood that the term “micropore” is used in the singular form for simplicity, but that the microporation devices described herein may form multiple artificial openings. Poration could reduce the barrier properties of a biological membrane into the body for selected purposes, or for certain medical or surgical procedures. For the purposes of this application, “poration” and “microporation” are used interchangeably and mean the same thing.

A “microporator” or “porator” is a component for a microporation device capable of microporation. Examples of a microporator or porator include, but are not limited to, a filament capable of conductively delivering thermal energy via direct contact to a biological membrane to cause the ablation of sonic portion of the membrane deep enough to form a micropore, an optically heated topical dye/absorber layer, an electromechanical actuator, a microlancet, an array of microneedles or lancets, a sonic energy ablator, a laser ablation system, a high-pressure fluid jet puncturer, and the like. As used herein, “microporator” and “porator” are used interchangeably.

As used herein, “penetration enhancement” or “permeation enhancement” means an increase in the permeability of the biological membrane to a drug, bio-active composition, or other chemical molecule, compound, particle or substance (also called “permeant”), so as to increase the rate at which the drug, bio-active composition, or other chemical molecule, compound or particle permeates the biological membrane.

As used herein, “enhancer,” “chemical enhancer,” “penetration enhancer,” “permeation enhancer,” and the like includes all enhancers that increase the flux of a permeant, analyte, or other molecule across the biological membrane, and is limited only by functionality. In other words, all cell envelope disordering compounds and solvents and any other chemical enhancement agents are intended to be included. Additionally, all active force enhancer technologies such as the application of sonic energy, mechanical suction, pressure, or local deformation of the tissues, iontophoresis or electroporation are included. One or more enhancer technologies may be combined sequentially or simultaneously. For example, a chemical enhancer may first be applied to permealize the capillary wall and then an iontophoretic or sonic energy field may be applied to actively drive a permeant into those tissues surrounding and comprising the capillary bed.

As used herein, “transdermal” means passage of a permeant into and through the biological membrane.

As used herein, the term “permeant,” “drug,” “permeant composition,” or “pharmacologically active agent” or any other similar term are used interchangeably to refer to any chemical or biological material or compound suitable for transdermal administration by the methods previously known in the art and/or by the methods taught in the present description, that induces a desired biological or pharmacological effect, which may include but is not limited to (1) having a prophylactic effect on the organism and preventing an undesired biological effect such as an infection, (2) alleviating a condition caused by a disease, for example, alleviating pain or inflammation, and/or (3) either alleviating, reducing, or completely eliminating the disease from the organism. The effect may be local, such as providing for a local anesthetic effect, or it may be systemic. Such substances include broad classes of compounds normally delivered into the body, including through body surfaces and membranes, including skin. In general, for example and not meant to be limiting, such substances can include any bioactive agents such as drug, chemical, or biological material that induces a desired biological or pharmacological effect. To this end, in one aspect, the permeant can be a small molecule agent. In another aspect, the permeant can be a macromolecular agent. In general, and without limitation, exemplary permeant include, but are not limited to, anti-infectives such as antibiotics and antiviral agents; analgesics and analgesic combinations; anorexics; antihelminthics; antiarthritics; antiasthmatic agents; anticoagulant; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral, and cerebral; central nervous system stimulants; vasoconstrictors; cough and cold preparations, including decongestants; hormones such as estradiol and other steroids, including corticosteroids; hypnotics; immunosuppressives; muscle relaxants; parasympatholytics; psychostimulants; sedatives; and tranquilizers.

The devices and methods of the instant description can also be used to transdermally deliver peptides, polypeptides, proteins, or other macromolecules known to be difficult to convey across the skin with existing conventional techniques because of their size. These macromolecular substances typically have a molecular weight of at least about 300 Daltons, and more typically, in the range of about 300 to 40,000 Daltons. Examples of polypeptides and proteins which may be delivered in accordance with the present description include, without limitation, antibodies, LHRH, LHRH analogs (such as goserelin, leuprolide, buserelin, triptorelin, gonadorelin, napharelin and leuprolide), GHRH, GHRF, insulin, insulinotropin, calcitonin, octreotide, endorphin, TRH, NT-36 (chemical name: N-[[(s)-4-oxo-2-azetidiny1]-carbony1]-L-histidy1-L-prolinamide), liprecin, pituitary hormones (e.g., HGH, HMG, HCG, desmopressin acetate, etc.), follicle luteoids, alpha-ANF, growth factor such as releasing factor (GFRF), beta-MSH, GH, somatostatin, bradykinin, somatotropin, platelet-derived growth factor, asparaginase, bleomycin sulfate, chymopapain, cholecystokinin, chorionic gonadotropin, corticotropin (ACTH), erythropoietin, epoprostenol (platelet aggregation inhibitor), glucagon, hirudin and hirudin analogs such as hirulog, hyaluronidase, interleukin-2, menotropins (urofollitropin (FSH) and LH), oxytocin, streptokinase, tissue plasminogen activator, urokinase, vasopressin, desmopressin, ACTH analogs, ANP, ANP clearance inhibitors, angiotensin II antagonists, antidiuretic hormone agonists, antidiuretic hormone antagonists, bradykinin antagonists, CD4, ceredase, CSI's, enkephalins, FAB fragments, IgE peptide suppressors, IGF-1, neurotrophic factors, colony stimulating factors, parathyroid hormone and agonists, parathyroid hormone antagonists, prostaglandin antagonists, cytokines, lymphokines, pentigetide, protein C, protein S, renin inhibitors, thymosin alpha-1, thrombolytics, TNF, GCSF, EPO, PTH, heparin having a molecular weight from 3000 to 12,000 Daltons, vaccines, vasopressin antagonist analogs, interferon-alpha, -beta, and -gamma, alpha-1 antitrypsin (recombinant), and TGF-beta genes; peptides; polypeptides; proteins; oligonucleotides; nucleic acids; and polysaccharides.

Further, as used herein, “peptide”, means peptides of any length and includes proteins. The terms “polypeptide” and “oligopeptide” are used herein without any particular intended size limitation, unless a particular size is otherwise stated. Exemplary peptides that can be utilized include, without limitation, oxytocin, vasopressin, adrenocorticotrophic hormone, epidermal growth factor, prolactin, luliberin or luteinising hormone releasing hormone, growth hormone, growth hormone releasing factor, insulin, somatostatin, glucagon, interferon, gastrin, tetragastrin, pentagastrin, urogastroine, secretin, calcitonin, enkephalins, endorphins, angiotensins, renin, bradykinin, bacitracins, polymixins, colistins, tyrocidin, gramicidines, and synthetic analogues, modifications and pharmacologically active fragments thereof, monoclonal antibodies and soluble vaccines. It is contemplated that the only limitation to the peptide or protein drug which may be utilized is one of functionality.

Examples of peptide and protein drugs that contain one or more amino groups include, without limitation, anti-cancer agents, antibiotics, anti-emetic agents, antiviral agents, anti-inflammatory and analgesic agents, anesthetic agents, anti-ulceratives, agents for treating hypertension, agents for treating hypercalcemia, agents for treating hyperlipidemia, etc., each of which has at least one primary, secondary or tertiary amine group in the molecule, preferably, peptides, proteins or enzymes such as insulin, calcitonin, growth hormone, granulocyte colony-stimulating factor (G-CSF), erythropoietin (EPO), bone morphogenic protein (BMP), interferon, interleukin, platelet derived growth factor (PDGF), vascular endothelial growth factor (VEGF), fibroblast growth factor (FGF), nerve growth factor (NGF), urokinase, etc. can be mentioned. Further examples of protein drugs include, without limitation, insulin, alpha-, beta-, and gamma-interferon, human growth hormone, alpha- and beta-1-transforming growth factor, granulocyte colony stimulating factor (G-CSF), granulocyte macrophage colony stimulating factor (G-MCSF), parathyroid hormone (PTH), human or salmon calcitonin, glucagon, somatostatin, vasoactive intestinal peptide (VIP), and LHRH analogs.

As used herein, an “effective” amount of a pharmacologically active agent means an amount sufficient to provide the desired local or systemic effect and performance at a reasonable benefit/risk ratio attending any medical treatment. An “effective” amount of a permeation or chemical enhancer as used herein means an amount selected so as to provide the desired increase in biological membrane permeability, the desired depth of penetration, rate of administration, and amount of drug delivered.

In various embodiments, transdermal permeant delivery systems and. methods that may be used and/or adapted for use with the compositions and methods described herein are described in one or more of U.S. Pat. Nos. 6,022,316, 6,142,939, 6,173,202, 6,183,434, 6,508,785, 6,527,716, 6,692,456, 6,730,028, 7,141,034, 7,392,080, 7,758,561, 8,016,811, 8,116,860, and/or 9,498,609, all of which are hereby incorporated by reference in their entireties and particularly for the purpose of describing such systems and methods. In various embodiments, the transdermal permeant delivery systems commercially available from Nitto Denko Corporation under the PASSPORT tradename may be used or adapted for use in delivering the permeant compositions described herein.

### Compositions

Various embodiments provide a composition for delivery of an active permeant through a pathway in a biological membrane of a subject comprising at least one thin solid tablet having an area density of more than 30 mg/cm² and less than 400 mg/cm². The thin solid tablet comprises at least one permeant, and at least a portion of the permeant is soluble in biological moisture received from at least one pathway formed through the biological membrane of the subject. In the pharmaceutical arts a tablet is typically defined as a pharmaceutical oral dosage form. Surprisingly, however, it has now been found that thin solid tablets as described herein are a safe, effective and convenient form by which a permeant (e.g., a pharmacologically active agent) can be provided for administration to a subject using a transdermal permeant delivery system as described elsewhere herein.

A large number of drugs have been formulated in tablet form, but their sizes and shapes have generally been selected to be relatively compact pill or capsule configurations suitable for safe and effective administration of the orally administrable drugs contained therein. In contrast, drugs intended for transdermal administration have generally been formulated as gels or flowable liquid forms (e.g., as solutions or dispersions) suitable for inclusion in a patch, such as those described in U.S. Pat. No. 9,498,609 and U.S. Patent Publication No. 2012/0238942, or in the form of powders printed onto a backing liner (see, e.g., U.S. Patent Publication No. 2004/0137044). Those skilled in the art have not been motivated to formulate drugs in the form of thin solid tablets having a relatively large area density as described herein because they would have been considered unsuitable and/or inferior to traditional compact pill and capsule forms for oral administration. In addition, various embodiments of thin solid tablet forms as described herein would have been considered undesirably prone to breakage as compared to flowable liquid forms typically used in transdermal patches, and thus inferior from a manufacturing, shipping and/or patient acceptance perspective. Various embodiments of thin solid tablet forms as described herein would also have been considered more difficult to administer orally, and thus undesirable for achieving patient acceptance and/or compliance as compared to relatively compact pill or capsule forms.

As used herein in the context of describing thin solid tablets suitable for delivery of a permeant through a pathway in a biological membrane of a subject, the term “tablet” refers to a form that would otherwise be considered a pharmaceutical oral dosage form consistent with the ordinary meaning of “tablet” as understood by those of skill in the pharmaceutical arts, but which has an area density greater than considered desirable for oral administration, Thin solid tablets can be in various wafer- or plate-like shapes such as elliptical, circular, triangular, rectangular, square, pentagonal, hexagonal, irregular, etc. In various embodiments thin solid tablets are substantially flat. In an embodiment, a substantially flat thin solid tablet is slightly curved or bowed to a degree that facilitates handling, e.g., as compared to a flat thin solid tablet that is more difficult to pick up from a flat surface.

In various embodiments, a thin solid tablet as described herein has an area density of more than 30 mg/cm², more than 40 mg/cm², more than 50 mg/cm², more than 60 mg/cm², more than 70 mg/cm², more than 80 mg/cm², more than 90 mg/cm², or more than 100 mg/cm²; less than 400 mg/cm², less than 350 mg/cm², less than 300 mg/cm², less than 250 mg/cm², or less than 200 mg/cm²; or in any range having endpoints defined by any two of the aforementioned values. For example, in various embodiments, the thin solid tablet has an area density of more than 30 mg/cm² and less than 400 mg/cm²; more than 40 mg/cm² and less than 400 mg/cm²; or more than 30 mg/cm² and less than 400 mg/cm².

In various embodiments, a thin solid tablet as described herein has a thickness (depending on the area density and the area of a face) of about 0.01 mm or greater, about 0.02 mm or greater, about 0.03 mm or greater, about 0.04 mm or greater, about 0.05 mm or greater, about 0.05 mm or greater, about 0.1 mm or greater, about 0.2 mm or greater, about 0.5 mm or greater, or about 1 mm or greater; about 10 mm or less, about 5 mm or less; about 2 mm or less; or about 1 mm or less; or in any range having endpoints defined by any two of the aforementioned values. For example, in various embodiments, the thin solid tablet has a thickness in the range of about 0.01 mm to about 10 mm or in the range of about 0.1 mm to about 5 mm.

In various embodiments, the thin solid tablet has a face in a manner analogous to the front or back face of a coin. In various embodiments, a face of the thin solid tablet has an area of about 0.01 cm² or greater, about 0.05 cm² or greater, about 0.1 cm² or greater, about 0.25 cm² or greater, about 0.5 cm² or greater, about 0.75 cm² or greater, or about 1 cm² or greater; or about 50 cm² or less, about 25 cm² or less, about 15 cm² or less, about 10 cm² or less, about 5 cm² or less, or about 2 cm² or less, or in any range having endpoints defined by any two of the aforementioned values. For example, in various embodiments, a face of a thin solid tablet has an area in the range of about 0.01 cm₂ to about 25 cm², about 0.1 cm² to about 10 cm², or about 0.15 cm₂ to about 5 cm².

A thin solid tablet as described can be made using various tableting materials and methods known to those skilled as adapted to the tablet configurations described herein. Such adaptations can be readily made by those of skill in the art in view of the guidance provided herein. In various embodiments the thin solid tablet comprises one or more excipients selected from: a binding agent, a disintegrating agent, a lubricant, a permeation enhancer, a solubilizer, an absorption control agent, an osmotic agent, a pH control agent, an antimicrobial agent, a release control agent, and a filler. For example, in various embodiments excipient is selected from one or more of sucrose, lactose, HP-β-CD, citric acid monohydrate, SBE-β-CD, ascorbic acid, urea, magnesium stearate, methylparaben, propylparaben, and Tween80.

The thin solid tablet also comprises one or more permeants as described elsewhere here. For example, in an embodiment the permeant is a hydrophobic drug. In an embodiment, the permeant has a water solubility that is less than 10 mg/mL. In an embodiment, the permeant comprises a high dose drug that requires a daily dosage at a rate that is difficult to achieve by typical transdermal patches in the absence of microporation. In an embodiment, the high dose drug requires an intake or more than 20 mg/day. In various embodiments, the permeant is selected from methylnaltrexone bromide, aripiprazole, sumatriptan succinate, exenatide, salts thereof, and combinations thereof. The permeant may be distributed throughout the thin solid tablet or concentrated in a particular region or regions. For example, in an embodiment the thin solid tablet comprises the permeant in the form of a layer on the tablet, in the form of a dispersion within the tablet, or a combination thereof. In an embodiment, the distribution is selected to control the rate of release of the permeant from the tablet and thereby provide deliver of the permeant a pathway in a biological membrane of a subject in a controlled manner, e.g., delayed release or sustained release.

In various embodiments, the permeant is one or more of: a small molecule drug, a peptide, a protein, an oligonucleotide, an antibody, a polysaccharide, and a vaccine. The one or more excipients in the thin solid tablet can be selected based on the characteristics of the permeant and the desired tablet configuration using routine experimentation guided by the detailed teachings provided herein. For example, in an embodiment the permeant is a hydrophobic drug and the excipient comprises an effective amount of a permeation enhancer for the hydrophobic drug. In various embodiments, the thin solid tablet comprises a solubilizer. The solubilizer can be selected based on the characteristics of the permeant and the degree of enhanced solubization desired. For example, in an embodiment the solubilizer is one or more of: a polyethylene glycol, a surfactant, a pH control agent, a cyclodextrin, a fatty acid and a salt of a fatty acid.

A composition for delivery of a permeant through a pathway in a biological membrane of a subject may be configured in various ways. For example, in an embodiment the composition includes a single thin solid tablet; in an alternate embodiment it comprises two or more thin solid tablets.

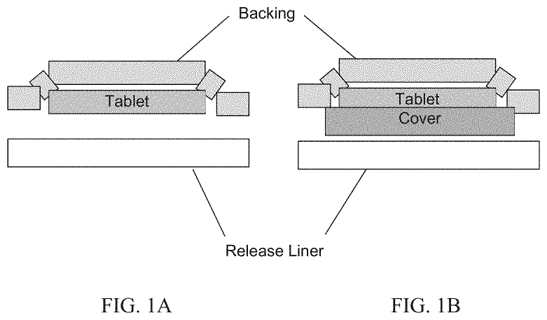

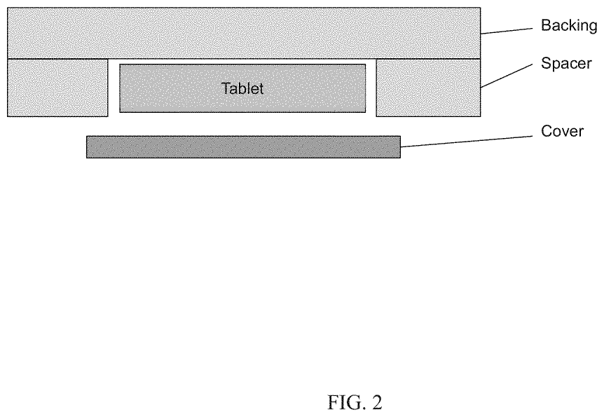

In an embodiment the thin solid tablet(s) is(are) incorporated into a patch. For example, an embodiment provides a patch for delivering an agent via at least one formed pathway through a biological membrane of a subject, the patch comprising a composition for delivery of a permeant through a pathway in a biological membrane of a subject that comprises a thin solid tablet as described herein. Thus, for example, the thin solid tablet in the patch can comprise a bioactive agent as described herein. FIGS. 1A, 1B and 2-4 illustrate various patch configurations.

In various embodiments, the patch is suitable for use in combination with a microporation device that is configured to form a pathway in a biological membrane of a subject. Transdermal permeant delivery systems that include suitable microporation devices are commercially available from Nitto Denko Corporation under the PASSPORT tradename. The PASSPORT system comprises a reusable handheld applicator and a single-use porator that can be used in combination with the patches described herein. Pressing the activation button of the applicator releases a pulse of energy to the porator. The rapid conduction of this energy into the surface of the skin painlessly ablates the stratum corneum under each filament to create the microchannels. A patch can then be applied to the ablated skin. Biological moisture from the subject passes through the formed microchannels and into the thin solid tablet(s) in the patch, solubilizing the drug and allowing it to pass through the skin via the microchannels and into the body of the subject.

An embodiment provides a method of treating a patient comprising:

opening at least one channel in the patient's skin;

applying a patch as described herein to the patient's skin to thereby contact the at least one thin tablet with the channel; and maintaining the at least one thin tablet in contact with the patient's skin for a period of time effective to:

(a) at least partially dissolve the permeant in biological moisture received from the pathway; and

(b) deliver a therapeutically effective amount of the resulting dissolved permeant through the pathway to the patient.

### EXAMPLES

Various embodiments and alternatives disclosed in further detail in the following examples, which are not in any way intended to limit the scope of the claims.

### Example 1

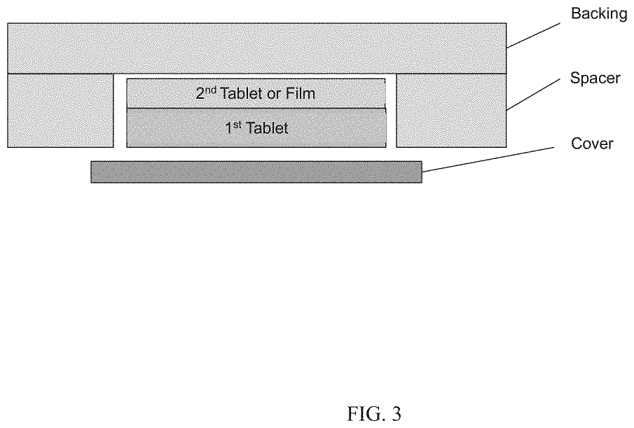

A series of thin solid tablets containing methylnaltrexone bromide (MNTX-Br) as active ingredient along with the other ingredients described in Table 1, were prepared using standard techniques for forming tablets. The tablets were 8 mm×8 mm square with a axial area of about 0.64 cm² and a tablet weight of 34.3 mg/cm² (22 mg/0.64 cm₂). Patches having the configuration illustrated in FIG. 3 were made using the thin solid tablets and applied to the skin of rats using a PASSPORT reusable handheld applicator and a single-use porator. PK data was collected in the usual manner. A dry patch (dispensing type) containing the same amount of MNTX-Br and the ingredients set forth in Table 2 below was used for comparison.

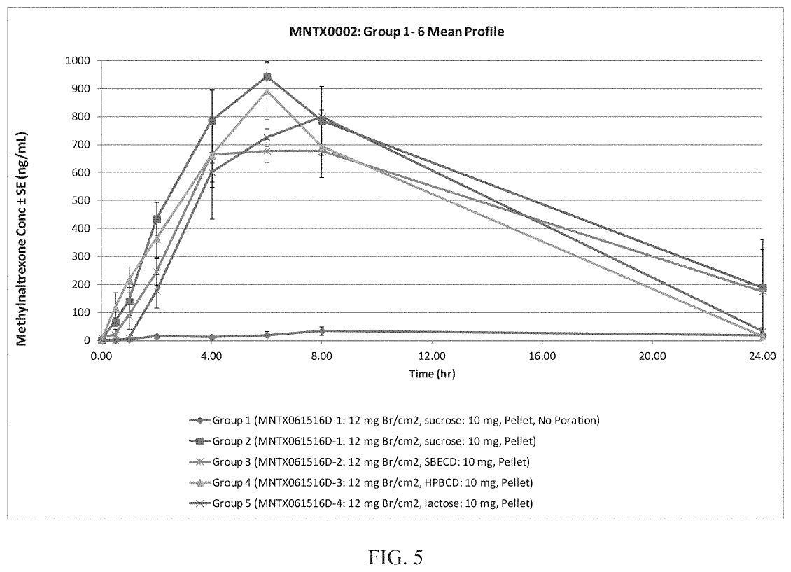

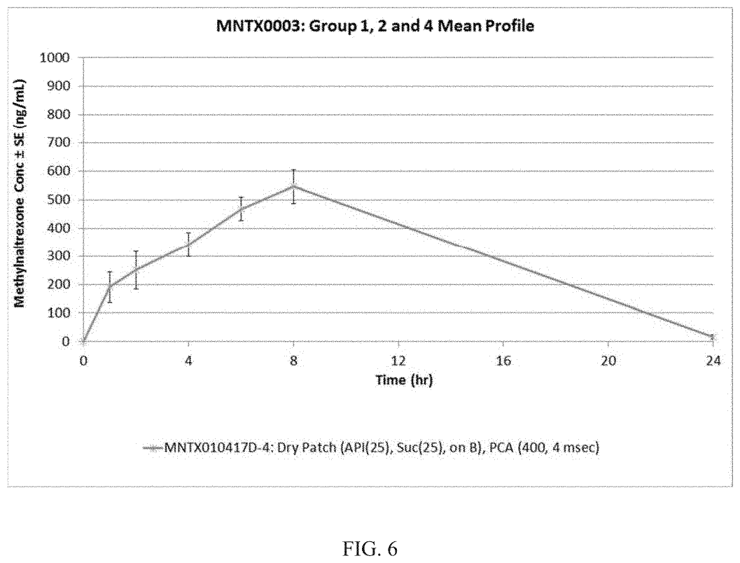

A summary of the resulting PK data is provided in Table 3. FIG. 5 illustrates the PK profiles of methylnaltrexone bromide released from thin solid tablets in the patches, and the comparative PK profile of methylnaltrexone bromide released from the dry patch is shown in FIG. 6. The amount of methylnaltrexone bromide released from the comparative patch was much less than the amounts released using the patches containing thin solid tablets as summarized in Table 3.

TABLE 1

| | AUC | BA | Cmax | Tmax |
|---|---|---|---|---|
| Group | (ng/ml * hr) | (%) | (ng/mL) | (hr) |
| 1 | 545.30 | 8.77 | 34.68 | 6.13 |
| 2 | 12831.26 | 206.47 | 996.75 | 6.00 |
| 3 | 10633.28 | 171.10 | 847.33 | 7.00 |
| 4 | 10226.79 | 164.56 | 900.40 | 6.50 |
| 5 | 10380.45 | 167.03 | 806.75 | 7.5 |

TABLE 2

| | AUC | BA | Cmax | Tmax |
|---|---|---|---|---|
| Group | (ng/ml * hr) | (%) | (ng/mL) | (hr) |
| 4 | 7227.2 | 55.8 | 561.9 | 7.3 |

TABLE 3

| Group | 002-G1 | 002-G2 | 002-G3 | 002-G4 | 002-G5 | 003-G4 |
|---|---|---|---|---|---|---|
| MNTX-Br (mg) | 12 | 12 | 12 | 12 | 12 | 25 |
| Sucrose (mg) | 10 | 10 | — | — | — | 25 |
| Lactose (mg) | — | — | — | — | 10 | — |
| SBECD (mg) | — | — | 10 | — | — | — |
| HPBCD (mg) | — | — | — | 10 | — | — |
| Total Solid (mg) | 22 | 22 | 22 | 22 | 22 | 50 |
| Axial section area | 0.64 | 0.64 | 0.64 | 0.64 | 0.64 | 1.00 |
| (cm²) | | | | | | |
| Tablet weight | 34.3 | 34.3 | 34.3 | 34.3 | 34.3 | Dry patch |
| (mg/cm²) | | | | | | |
| Poration; filament | No | 400, 4 ms | 400, 4 ms | 400, 4 ms | 400, 4 ms | 400, 4 ms |
| density per cm², | poration | | | | | |
| pulse length (ms) | | | | | | |
| AUC (ng/mL*hr) | 545 | 12831 | 10633 | 10227 | 10380 | 7227.2 |
| rBAsc (%) | 8.77 | 206.47 | 171.10 | 164.56 | 167.03 | 55.8 |
| Cmax (ng/mL) | 35 | 997 | 847 | 900 | 807 | 561 |
| Tmax (hr) | 6.1 | 6.0 | 7.0 | 6.5 | 7.5 | 7.3 |

*Osmotic agent: Sucrose, Lactose, SBECD, HPBCD

### Example 2

A series of thin solid tablets containing Aripiprazole as active ingredient along with the other ingredients described in Table 4. were prepared using standard techniques for forming tablets. The tablets were 9 mm×9 mm square with an axial area of about 0.81 cm² and a tablet weight of 61.7 mg/cm² (50 mg/0.81 cm²). Patches having the configuration illustrated in FIG. 3 were made using the thin solid tablets and applied to the skin of rats using a PASSPORT reusable handheld applicator and a single-use porator. PK data was collected in the usual manner.

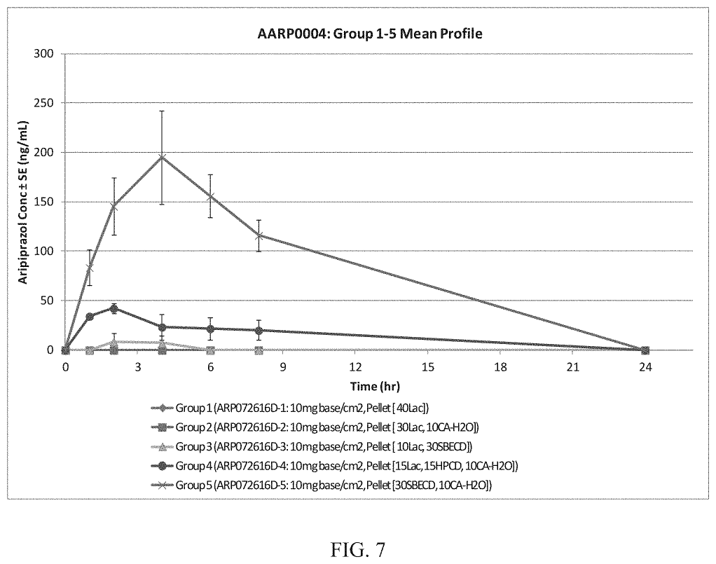

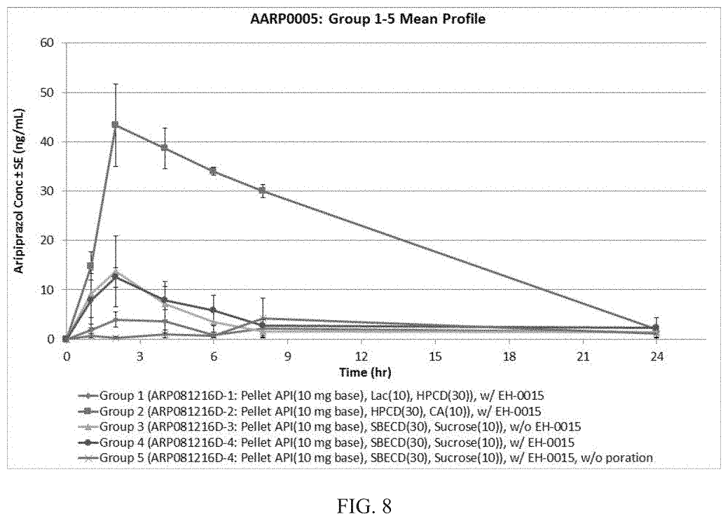

A summary of the resulting PK data is provided in Table 5 and FIGS. 7 and 8 illustrates the PK profiles of aripiprazole released from the patches.

TABLE 4

| | 004 | 004 | 004 | 004 | 004 | 005 | 005 | 005 | 005 | 005 |
|---|---|---|---|---|---|---|---|---|---|---|
| Group | #1 | #2 | #3 | #4 | #5 | #1 | #2 | #3 | #4 | #5 |
| Aripiprazol | 10.00 | 10.00 | 10.00 | 10.00 | 10.00 | 10.00 | 10.00 | 10.00 | 10,00 | 10.00 |
| (mg) | | | | | | | | | | |
| Lactose (mg) | 40.00 | 30.00 | 10.00 | 15.00 | | 10.00 | | | | |
| SBECD (mg) | | | 30.00 | | 30.00 | | | 30.00 | 30.00 | 30.00 |
| HPCD (mg) | | | | 15.00 | | 30.00 | 30.00 | | | |
| Citric acid | | 10.00 | | 10.00 | 10.00 | | 10.00 | 10.00 | 10.00 | 10.00 |
| monohydrate | | | | | | | | | | |
| (mg) | | | | | | | | | | |
| Total (mg) | 50.00 | 50.00 | 50.00 | 50.00 | 50.00 | 50.00 | 50.00 | 50.00 | 50.00 | 50.00 |
| Patch Type | With | With | With | With | With | With | With | No | With | With |
| | cover | cover | cover | cover | cover | cover | cover | cover | cover | cover |
| Poration: | 400, | 400, | 400, | 400, | 400, | 400, | 400, | 400, | 400, | no |
| filament | 4 ms | 4 ms | 4 ms | 4 ms | 4 ms | 4 ms | 4 ms | 4 ms | 4 ms | |
| density per | | | | | | | | | | |
| cm², pulse | | | | | | | | | | |
| length (ms) | | | | | | | | | | |

*Osmotic agent: lactose, SBECD, HPCD
** Solubilizer: SBECD, HPCD and CA

TABLE 5

| | AUC | Cmax | Tmax |
|---|---|---|---|
| Group | (ng/ml * hr) | (ng/mL) | (hr) |
| 004_#1 | 0.0 | 0.0 | 0.0 |
| 004_#2 | 0.0 | 0.0 | 0.0 |
| 004_#3 | 27.2 | 8.6 | 0.7 |
| 004_#4 | 367.2 | 42.2 | 2.0 |
| 004_#5 | 2046.4 | 200.9 | 4.7 |
| 005_#l | 47.1 | 6.4 | 4.7 |
| 005_#2 | 510.6 | 46.7 | 2.7 |
| 005_#3 | 76.7 | 13.7 | 1.3 |
| 005_#4 | 97.1 | 13.5 | 1.7 |
| 005_#5 | 51.6 | 4.9 | 4.0 |

### Example 3

A series of thin solid tablets containing Aripiprazole as active ingredient along with the other ingredients described in Table 6, were prepared using standard techniques for forming tablets. The tablets were 9 mm×9 mm square with an axial area of about 0.81 cm² and a tablet weights of 61.7 mg/cm² and 98 mg/cm² (50 mg/0.81 cm₂and 80 mg/0.81 cm², respectively). Patches having the configuration illustrated in FIG. 3 were made using the thin solid tablets and applied to the skin of rats using a PASSPORT reusable handheld applicator and a single-use porator. PK data was collected in the usual manner.

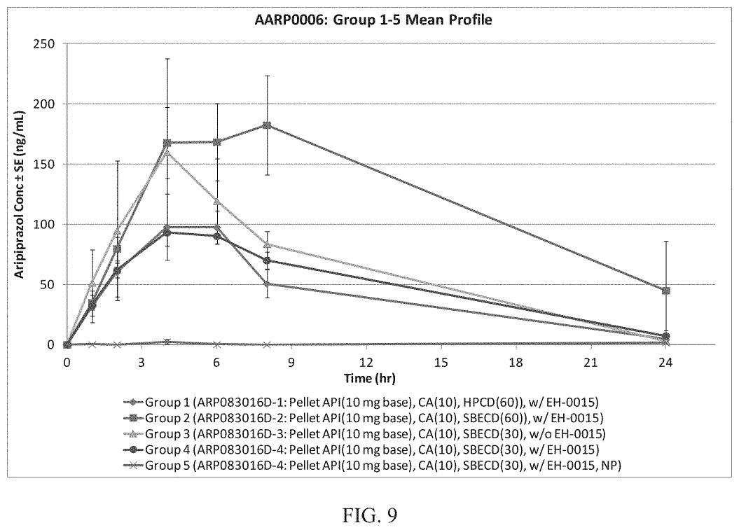

A summary of the resulting PK data is provided in Table 7 and FIG. 9 illustrates the PK profiles of aripiprazole released from the patches.

TABLE 6

| Group | #1 | #2 | #3 | #4 | #5 |
|---|---|---|---|---|---|
| Aripiprazol (mg) | 10 | 10 | 10 | 10 | 10 |
| Lactose (mg) | — | — | — | — | — |
| SBECD (mg) | — | 60 | 30 | 30 | 30 |
| HPCD (mg) | 60 | — | — | — | — |
| Citric acid | 10 | 10 | 10 | 10 | 10 |
| monohydrate | | | | | |
| (mg) | | | | | |
| Total (mg) | 80 | 801 | 50 | 50 | 50 |
| Patch Type | F | F | E | F | F |
| Poration: | 400 | 400 | 400 | 400 | — |
| filament | | | | | |
| density per | | | | | |
| cm² | | | | | |
| Pulse length | 4 | 4 | 4 | 4 | — |
| (ms) | | | | | |
| AUC | 1004 | 2829 | 1528 | 1181 | 19 |
| (ng/mL * hr) | | | | | |
| Cmax | 114 | 204 | 162 | 95 | 4 |
| (ng/mL) | | | | | |
| Tmax | 5.0 | 6.01 | 5.3 | 5.0 | 11.3 |
| (hr) | | | | | |

TABLE 7

| | AUC | Cmax | Tmax |
|---|---|---|---|
| Group | (ng/ml * hr) | (ng/mL) | (hr) |
| 1 | 1003.7 | 113.7 | 5.0 |
| 2 | 12829.3 | 204.2 | 6.0 |
| 3 | 11527.9 | 162.4 | 5.3 |
| 4 | 1181.1 | 94.8 | 5.0 |
| 5 | 19.1 | 3.7 | 11.3 |

### Example 4

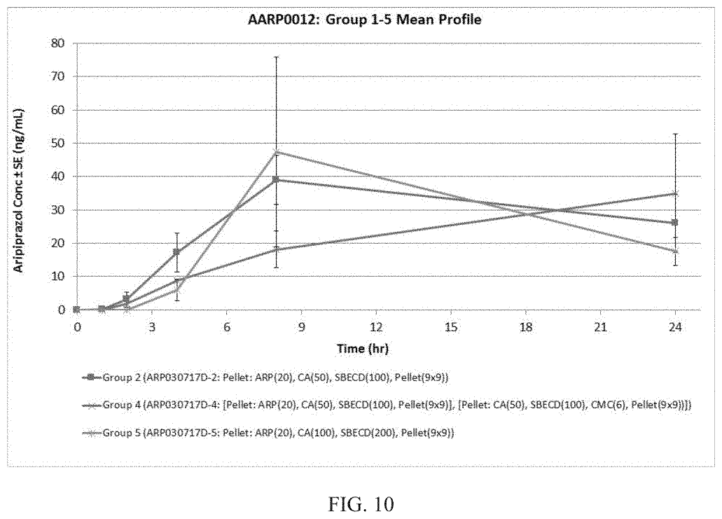

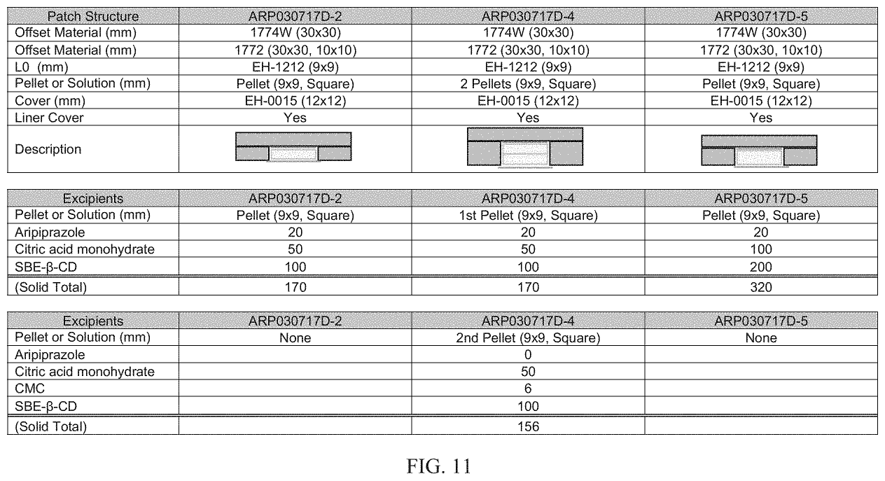

A series of thin solid tablets containing Aripiprazole as active ingredient along with the other ingredients described in FIG. 11, were prepared using standard techniques for forming tablets. The tablets were 9 mm×9 mm square with an axial area of about 0.81 cm² and a tablet weights of 210.0 mg/cm², 402.5 mg/cm² and 395.1 mg/cm² (170 mg/0.81 cm², 326 mg/0.81 cm² and 320 mg/0.81 cm², respectively). Patches having the configuration illustrated in FIGS. 3 and 11 were made using the thin solid tablets and applied to the skin of hairless Guinea pigs using a PASSPORT reusable handheld applicator and a single-use porator. PK data was collected in the usual manner. FIG. 10 illustrates the PK profiles of aripiprazole released from the patches, demonstrating sustained release.

### Example 5 (Comparative)

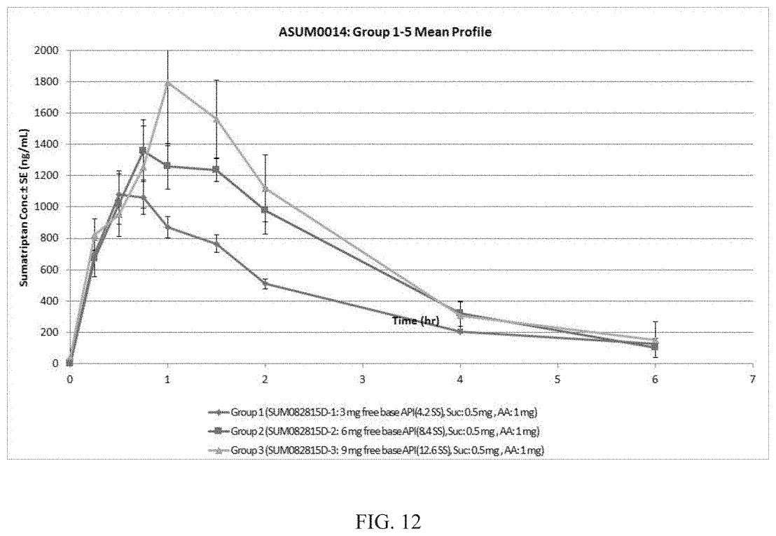

A series of immediate release dry patches containing sumatriptan as active ingredient along with the other ingredients described in Table 8, were prepared. The immediate release dry patches were applied to the skin of hairless Guinea pigs and PK data was collected in the usual manner. A summary of the resulting PK data is provided in Table 9 and FIG. 12 illustrates the PK profiles of sumatriptan released from the patches. Color changes in the ingredients of the immediate release patches were observed to occur during storage, indicating a stability problem resulting from interaction between the sumatriptan and ascorbic acid.

TABLE 8

| | #1 | #2 | #3 |
|---|---|---|---|
| Sumatriptan succinate (mg) | 4.20 | 8.40 | 12.60 |
| Sucrose (mg) | 0.50 | 0.50 | 0.50 |
| Ascorbic acid (mg) | 1.00 | 1.00 | 1.00 |
| Total (mg) | 5.70 | 9.90 | 14.10 |
| Poration: filament density | 400, | 400, | 400, |
| per cm², pulse length (ms) | 4 ms | 4 ms | 4 ms |

* Osmotic agent: Sucrose
** Enhancer: Ascorbic Acid

TABLE 9

| | AUC | BA | Cmax | Tmax |
|---|---|---|---|---|
| Group | (ng/ml * hr) | (%) | (ng/mL) | (hr) |
| 1 | 2598.5 | 96.5 | 1035.3 | 0.8 |
| 2 | 3814.5 | 70.9 | 1404.9 | 1.3 |
| 3 | 4388.9 | 54.4 | 1582.6 | 11 |

### Example 6

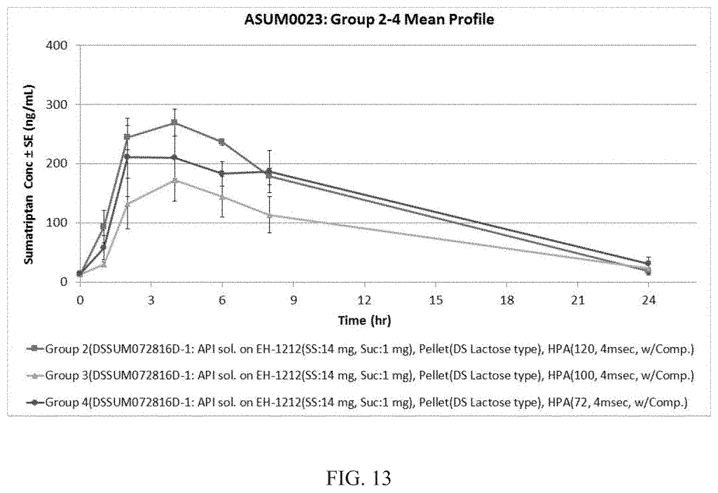

A series of thin solid tablets containing sumatriptan as active ingredient along with the other ingredients described in Table 10, were prepared using standard techniques for forming tablets. The tablets were 9 mm×9 mm square with an axial area of about 0.81 cm² and had tablet weights of 56.44 mg/cm² (45.72 mg/0.81 cm²). Patches having the configuration illustrated in FIG. 3 were made using the thin solid tablets and applied to the skin of hairless Guinea pigs using a PASSPORT reusable handheld applicator and a single-use porator. PK data was collected in the usual manner. A summary of the resulting PK data is provided in Table 11 and FIG. 13 illustrates the PK profiles of sumatriptan released from the patches. The stability issue observed in the comparative immediate release patches of Example 5 was not observed because the sumatriptan and ascorbic acid were separated.

TABLE 10
Film Layer (dispensed layer)

| L1:API Formulation | #1, 2, 3 | |
|---|---|---|
| Sumatriptan succinate (mg) | 14 | Active pharmaceutical |
| | | ingredient (API) |
| Sucrose (mg) | 1 | Osmotic agent |
| Total (mg) | 15 | — |

Tablet Layer

| Pellet-Excipients | #1, 2, 3 | |
|---|---|---|
| Anhydrous Lactose (mg) | 5.00 | Binder/osmotic agent |
| Urea (mg) | 24.00 | Enhancer |
| Ascorbic Acid (mg) | 16.00 | Enhancer/sustained agent |
| Magnesium Stearate (mg) | 0.50 | Lubricant |
| Methylparaben (mg) | 0.20 | Anti-microbial agent |
| Propylparaben (mg) | 0.02 | Anti-microbial agent |
| Total (mg) | 45.72 | — |
| Poration; fiament density per | 72, 120, | — |
| cm², pulse length (ms) | 120/4 ms | |

TABLE 11

| | AUC | BA | Cmax | Tmax |
|---|---|---|---|---|
| Group | (ng/ml * hr) | (%) | (ng/mL) | (hr) |
| 2.0 | 3233.0 | 36.0 | 279.3 | 4.0 |
| 3.0 | 2083.2 | 23.2 | 172.6 | 4.0 |
| 4.0 | 3094.7 | 34.5 | 244.3 | 4.0 |

### Example 7 (Comparative)

An immediate release dry patch containing exenatide as active ingredient along with the other ingredients described in Table 12, was prepared. Color changes in the ingredients of the immediate release patch was observed to occur during storage, indicating a stability problem resulting from interaction between the exenatide and ascorbic acid.

TABLE 12

| Excepients | #6 | |
|---|---|---|
| Exenatide (mg) | 0.4 | API |
| Sucrose (mg) | 8 | Osmotic agent |
| Urea (mg) | 8 | Enhancer |
| Ascorbic Acid (mg) | 2 | Enhancer/sustained agent |
| Tween 80 (mg) | 0.07 | Surfactant |
| Total (mg) | 18.47 | |

### Example 8

A thin solid tablet containing exenatide as active ingredient along with the other ingredients described in Table 13, was prepared using standard techniques for forming tablets. tablet was 9 mm×9 mm square with an axial area of about 0.81 cm² and had a tablet weight of 56.44 mg/cm² (45.72 mg/0.81 cm²). A patch having the configuration illustrated in FIG. 3 was made using the thin solid tablet, The stability issue observed in the comparative immediate release thy patch of Example 7 was not observed because the exenatide and ascorbic acid were separated.

TABLE 10
Film Layer (dispensed layer)

| 11: API Formulation | #6 | |
|---|---|---|
| Exenatide (mg) | 1 | API |
| Sucrose (mg) | 1 | Osmotic agent |
| Total (mg) | 2 | — |

Tablet Layer

| Pellet-Excipients | #6 | |
|---|---|---|
| Anhydrous Lactose (mg) | 5.00 | Binder/osmotic agent |
| Urea (mg) | 24.00 | Enhancer |
| Ascorbic Acid (mg) | 16.00 | Enhancer/sustained agent |
| Magnesium Stearate (mg) | 0.50 | Lubricant |
| Methylparaben (mg) | 0.20 | Anti-microbial agent |
| Propylparaben (mg) | 0.02 | Anti-microbial agent |
| Total (mg) | 45.72 | — |

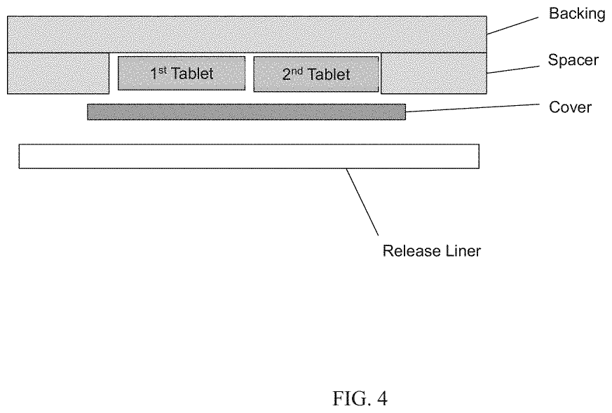

The data in the Examples above indicates that thin solid tablets as described herein are useful in a variety of demanding applications, particularly when used in combination with a suitable microporation devices such as those commercially available from Nitto Denko Corporation under the PASSPORT tradename. For example, in an embodiment, a patch containing a thin solid tablet as described herein has a relatively high loading of a hydrophobic drug, and thus can be used in the manner described herein to deliver the drug to a subject at a high dose of 20 mg/day or greater. Relatively large quantities of solubilizers are typically used to enhance the solubility of such drugs for use in a conventional transdermal delivery patch, thus limiting the drug loading and resulting daily dosage. In another embodiment, a patch containing two or more thin solid tablets as described herein (or a thin solid tablet haying a coating), e.g., as illustrated in FIGS. 3-4, enhances the ability of the patch to provide desirable PK profiles (such as controlled release) and/or enhances stability by enabling separation of ingredients that would otherwise interact in an undesirable manner, In another embodiment, a patch containing two or more thin solid tablets as described herein (or a thin solid tablet having a coating), e.g., as illustrated in FIGS. 3-4, enables the multiple active ingredients (e.g., drugs) to be delivered from a single patch, thereby facilitating the administration of combination therapies.

1. A composition for delivery of a permeant through a pathway in a biological membrane of a subject comprising:
at least one thin solid tablet having an area density of more than 30 mg/cm² and less than 400 mg/cm²;
wherein the thin solid tablet comprises at least one permeant; and
wherein at least a portion of the permeant is soluble in biological moisture received from at least one pathway formed through the biological membrane of the subject.

2. The composition of claim 1, wherein the thin solid tablet comprises one or more excipients selected from the group consisting of: a binding agent, a disintegrating agent, a lubricant, a permeation enhancer, a solubilizer, an absorption control agent, an osmotic agent, a pH control agent, an antimicrobial agent, a release control agent, and a filler.

3. The composition of claim 2, wherein the permeant comprises a drug and the excipient comprises an effective amount of a permeation enhancer for the drug.

4. The composition of claim 1, wherein the permeant is one or more selected from the group consisting of: a small molecule drug, a peptide, a protein, an oligonucleotide, an antibody, a polysaccharide, and a vaccine.

5. The composition of claim 3, wherein the permeant has a water solubility that is less than 10 mg/mL.

6. The composition of claim 3, wherein the permeant comprises a high dose drug.

7. The composition of claim 6, wherein the permeant requires an intake of more than 20 mg/day.

8. The composition of claim 2, wherein the solubilizer is selected from the group consisting of: a polyethylene glycol, a surfactant, a pH control agent, a cyclodextrin, a fatty acid and a salt of a fatty acid.

9. The composition of claim 1, wherein the thin solid tablet has a thickness in the range of about 0.01 mm to about 10 mm.

10. The composition of claim 1, wherein the thin solid tablet has a thickness in the range of about 0.1 mm to about 5 mm.

11. The composition of claim 1, wherein a face of the thin solid tablet has an area in the range of about 0.01 cm² to about 25 cm².

12. The composition of claim 1, wherein a face of the thin solid tablet has an area in the range of about 0.1 cm² to about 10 cm².

13. The composition of claim 1, wherein a face of the solid tablet has an area in the range of about 0.15 cm² to about 5 cm².

14. The composition of claim, wherein the thin solid tablet further comprises a second permeant.

15. The composition of claim 1, wherein the thin solid tablet comprises a permeant in the form of a layer.

16. The composition of claim 15, wherein the layer is on a face of the thin solid tablet.

17. The composition of claim 1, wherein the permeant is selected from the group consisting of methylnaltrexone bromide, aripiprazole, sumatriptan succinate, exenatide, salts thereof, and combinations thereof.

18. The composition of claim 1, wherein the excipient is selected from the group consisting of: sucrose, lactose, HP-β-CD, citric acid monohydrate, SBE-β-CD, ascorbic acid, urea, magnesium stearate, methylparaben, propylparaben, and Tween80.

19. The composition of claim 1, comprising at least two thin solid tablets.

20. A patch for delivering an agent via at least one formed pathway through a biological membrane of a subject, the patch comprising the composition of claim 1.

21. The patch of claim 20, wherein the at least one thin solid tablet comprises a bioactive agent.

22. The patch of claim 21, wherein the patch provides an immediate release profile and a sustained release profile of the permeant from the at least one thin solid tablet through the at least one pathway formed through the biological membrane of the subject.

23. The patch of claim 20, further comprising:
a tablet layer comprising the at least one thin solid tablet;
a backing layer over the tablet layer; and
a release liner layer under the tablet layer.

24. The patch of claim 23, further comprising a cover under the tablet layer and over the release liner layer, the cover being configured to reduce contact between the at least one thin solid tablet and the release liner layer.

25. The patch of claim 23, further comprising a spacer layer between the backing layer and the release liner layer, the spacer layer being laterally adjacent to the at least one thin solid tablet and configured to maintain a separation distance between the backing layer and the release liner layer, the separation distance being in the range of about 50% to about 150% of the thickness of the thin solid tablet.

26. The patch of claim 22, wherein the tablet layer comprises two or more thin solid tablets.

27. The patch of claim 22, further comprising an adhesive layer under the backing layer and over the release liner layer.

28. The patch of claim 27, wherein the adhesive layer is under the spacer layer.

29. A method of treating a patient comprising:
opening at least one channel in the patient's skin;
applying the patch of claim 20 to the patient's skin to thereby contact the at least one thin tablet with the channel; and
maintaining the at least one thin tablet in contact with the patient's skin for a period of time effective to:
(a) at least partially dissolve the permeant in biological moisture received from the pathway; and
(b) deliver a therapeutically effective amount of the resulting dissolved permeant through the pathway to the patient.

30. A method of delivering a permeant through a pathway in a biological membrane of a subject comprising applying the patch of claim 20 to the patient's skin.

31. A method of transdermal administration of a permeant comprising applying the patch of claim 20 to a dermal surface of a subject.

32. A transdermal drug delivery system for delivering a drug, comprising:
a transdermal microporation device configured to form a pathway through the skin of a subject; and
the patch of claim 20.

33. The transdermal drug delivery system for delivering a drug of claim 32, wherein the at least one thin tablet is configured to be in contact with the skin of the subject for a period to time effective to at least partially dissolve the permeant in biological moisture received from the pathway, and the at least one thin tablet is configured to deliver a therapeutically effective amount of the resulting dissolved permeant through the pathway to the patient.

34. The transdermal drug delivery system for delivering a drug of claim 32, wherein the patch of claim 20 is configured to be applied to a dermal surface of the subject.

35. (canceled)

36. (canceled)
